(19) United States
(12) Patent Application Publication
(10) Pub. No.: US 2006/0177412 A1
(43) Pub. Date: Aug. 10, 2006

(54) Deodorizer

(76) Inventors: Radhakrishnan Janardanan Nair, Kobe (JP); Ryohei Ohtani, Nishinomiya (JP); Mayumi Ashiya, Kobe (JP); Manivannan Kandasamy, Kobe (JP)

Correspondence Address: THE PROCTER & GAMBLE COMPANY;INTELLECTUAL PROPERTY DIVISION, WINTON HILL TECHNICAL CENTER - BOX 161, 6110 CENTER HILL AVENUE, CINCINNATI, OH, 45224, US

(21) Appl. No.: 11/101,721

(22) Filed: Apr. 8, 2005

Related U.S. Application Data
(60) Provisional application No. 60/651,781, filed on Feb. 10, 2005.

(51) Int. Cl. A61L 9/015; A61K 33/44; A61K 33/32

(52) U.S. Cl. 424/76.2; 424/125; 424/641

(57) ABSTRACT

A placement type deodorizer contains a malodor removing material, wherein the deodorizer has Malodor Accessibility Factor (MAF) of more than about 5×10⁴ ppm² m²/(g*min) and wherein the malodor removing material comprises a malodor removing active.

### CROSS REFERENCE TO RELATED APPLICATION

This application claims the benefit of U.S. Provisional Application No. 60/560,795, filed Apr. 8, 2004 and U.S. Provisional Application No. 60/651,781, filed Feb. 10, 2005.

### FIELD OF THE INVENTION

The present invention relates to a deodorizer. Specifically, the present invention relates to a placement type deodorizer.

### BACKGROUND OF THE INVENTION

There are many types of malodors in society. Especially, there are many uncomfortable malodors at home. For example, when foods are rotten, amine type or hydrogen sulfide type malodors may cause kitchens or refrigerators to smell unpleasant. Also, ammonium type or mercaptan type malodors may take toilets smell unpleasant. In order to deodorize or remove these malodors, many types of deodorizers have been developed and sold in the market.

These deodorizers have deodorant actives. One of the typical deodorant actives is plant extracts, for example, catechin or flavonoid. It is believed that plant extracts veil malodor molecules and as a result, malodors are removed. Another type of deodorant actives is a chemical compound which reacts with malodor molecules, such as chlorine dioxide, hypo chloride or ozone. These chemical compounds decompose malodor molecules by oxidization or reduction and thus, malodors are removed. In addition, some deodorant actives use a neutralizing reaction with malodors and as a result, malodors are removed.

These deodorizer actives are incorporated into deodorizer packages and provide malodor removal performance. These deodorizer packages are, mainly divided into two categories: a spray type and a placement type. Spray type generally contains liquid type deodorant actives. When users detect malodors, they spray it onto the air. In contrast, placement type generally contains gel or solid type deodorant actives and users put these deodorizers at places where malodors smell and/or are released such as kitchens or toilets.

However, these deodorizers do not always meet users' need because of insufficient malodor removal performance or too slow malodor removal. Some placement type deodorizers contain fan devices to create agitation of air for better malodor removal. However, fan devices increase the cost of the product and users need to change batteries which cause inconvenience and increased cost.

Therefore, there is a need to provide a deodorizer which solves these problems.

### SUMMARY OF THE INVENTION

The present invention relates to a placement type deodorizer having a malodor removing material. The deodorizer has Malodor Accessibility Factor (MAP) of more than about 5×10⁴ ppm²cm²/(g*min), preferably, from about 5×10⁴ ppm²cm²/(g*min) to 15×10⁷ ppm²cm²/(g*min)

The MAF consists of the parameters: Effective Open Surface Area (EOSA), Absorption Rate (AR) and Absorption Capacity (AC). The malodor removing material comprises a malodor removing active and can further comprise a carrier.

The present invention can provide an improved placement type deodorizer. Current placement type deodorizers without a battery are not always able to provide sufficient malodor performance because these deodorizers cannot provide air agitation. Thus, it is a common to use a fan device to create agitation of air for obtaining better malodor removal. However, fan devices increase the cost of the product and the user needs to change a battery which causes them inconvenience. To solve the problem, the present invention tries to increase the malodor removal performance of the deodorizer and finally reaches at focusing on parameters of ESOA, AR and AC. Thus, the present invention provides a placement type deodorizer having sufficient malodor removal performance without requiring battery-powered air agitation. As a result, users can enjoy the improved malodor performance without changing batteries.

Also, the deodorizer of the present invention provides such performance much more quickly than other products. For example, once users put the deodorizer at any place where malodors smell unpleasant, users typically notice malodor removal performance within the initial 10 minutes, a result which previous products cannot achieve.

### DETAILED DESCRIPTION OF THE INVENTION

All percentages, ratios and proportions herein are by weight of the composition, unless otherwise specified. All temperatures are in degrees Celsius (° C.) unless otherwise specified.

As used herein, the term “comprising” and its derivatives means are intended to be open ended terms that specify the presence of the stated features, elements, components, groups, integers, and/or steps, but do not exclude the presence of other, unstated features, elements, components, groups, integers, and/or steps. This definition also applies to words of similar meaning, for example, the term “have”, “include”, “be provided with” and their derivatives. This term encompasses the terms “consisting of” and “consisting essentially of”.

As used herein, the term “MAF (Malodor Accessibility Factor)” means a multiplication of factors consisting of EOSA (Effective Open Surface Area), AR (Absorption Rate) and AC (Absorption Capacity of the malodor removing active).

As used herein, the term “ESOA (Effective Open Surface Area)” means a factor which is decided by the effective open surface area of the malodor removing material (cm²). This factor can be varied by different means such as overall area of the device, partition or layers. However, EOSA is the area in which the malodor removing material may or can be accessible to an air.

In the absorption type, malodor molecules need to come in contact with the malodor removing material. This means that the more the absorption surface area, the more will be the malodor molecules come in contact with the surface and thereby increasing the efficacy of the deodorizer product. However, a large surface area means a large device which may be difficult to handle it as a home deodorizing device. In the present invention a design of the device which gives optimum surface area is defined.

However, placement type deodorizers usually include a tray which contains a malodor removing material and is covered with a lid. The lid is either a flat one which is in level with the tray mouth and has some holes or slits to ensure air circulation. In some other type, the lid is like a cap with certain height from the tray open area. The sides of the cap as well as the top may have some holes or slits for allowing air circulation. This does not effectively allow the air come to contact with the malodor removing material as much as having a complete open surface (without any lid—only opened tray). The EOSA is taken as follows: if the package lid open area is smaller than the tray open area, then the package lid open area is considered as EOSA. This is because the package lid open area is the limiting factor in the access of air on to the deodorizer. If both are equal or the package lid open area is higher than the tray open area, then the tray open area is considered as EOSA. This is because, in this case even though the package lid open area has very high area opened, the maximum area of deodorizer coming to contact with air is the tray open area.

In some cases in addition to the net or cap containing holes and attached to the tray, there may be a roof for the purpose of overall decoration of the device. In such cases the package lid open area is estimated without considering the roof.

As used herein, the term “AR” means the factor which is defined as a rate at which an ammonia gas (NH₃) is absorbed by the malodor removal material (ppm/min). A method for measuring AR is explained hereinafter.

As used herein, the term “AC” means a factor which is defined as a total amount of ammonia (NH₃) gas absorbed by a gram of the malodor removal material (ppm/g). A method for measuring AC is explained hereinafter.

The deodorizer of the present invention comprises a malodor removing material. Also, the deodorizer of the present invention has Malodor Accessibility Factor (MAF) of more than about 5×10⁴ ppm² cm²/(g*min) The malodor removing material comprises a malodor removing active and preferably comprises a carrier.

(1) Malodor Removing Material

The malodor removing material comprises a malodor removing active as an essential ingredient and preferably comprises a carrier. The malodor removing material can further comprise other ingredients such as perfume, dye, stabilizer, water, organic solvent, preservatives etc.

(a) Malodor Removing Active

The malodor removing active of the present invention is not limited and can be any materials as long as it provides the MAF of the present invention to the deodorizer. The malodor removing active is preferably selected from the group consisting of a polymer, a chlorine dioxide, a cyclodextrin, a titanium dioxide, a phtalocyanine, a zinc chloride, a copper compound, an iron compound, a reactive aldehyde, a plant extract, an activated carbon, a zeolite and a mixture thereof

Polymer

The polymer which can be used as the malodor removing active needs to have at least one functional group. The functional group has an ability to adsorb polar substances, for example, hydrophilic groups, cationically dissociating groups, or anionically dissociating groups. Preferably, the polymer for the malodor removing active of the present invention has more than one functional group selected from the group consisting of hydrophilic groups, cationically dissociating groups, anionically dissociating groups and a mixture thereof.

The polymer of the present invention is effective for malodor removal. It is believed that the backbone of the polymer where the functional group attaches onto provides an open structure for the functional group. As a result, these polymers ensure easy accessibility to a malodor molecule. This type of structure differentiates it from other polymers with similar functional groups.

Examples of such hydrophilic groups include a hydroxyl group, a hydroxyalkyl group, an amino group and a pyrrolidonyl group. Preferred hydrophilic groups include a hydroxyl group, a C₂-C₁₀ hydroxyalkyl group and a pyrrolidonyl group. One or more hydrophilic group may be introduced into the polymer.

The term “cationically dissociating groups” as used herein means that their ion-exchange groups whose counter ion is a cation. A typical cationically dissociating group is an acid group. Cationically dissociating groups have the ability to adsorb polar substances and are capable of releasing a proton (hydrogen ion) to enter into neutralizing reaction with basic substances, such as ammonia or amines. As a result, the basic substances can be removed. One or more cationically dissociating groups may be introduced into the polymer.

Examples of such cationically dissociating groups include a carboxyl group, a sulfate group, a phosphate group, a sulfoethyl group, a phosphomethyl group and a carbomethyl group. Preferred cationically dissociating groups include a sulfate group and a carboxyl group.

The term “anionically dissociating groups” as used herein means that those ion-exchange groups whose counter ion is an anion. Therefore, anionically dissociating groups have the ability to absorb polar substances and are capable of entering into neutralizing reaction with acidic substances, such as, hydrogen sulfide or mercaptans. As a result, the acidic substances can be removed. One or more kinds of anionically dissociating substances may be introduced into the polymer.

Examples of such anionically dissociating groups include a quaternary ammonium group and amino groups. The amino groups include primary, secondary and tertiary amino groups, for example, an amino group, a methylamino group, a dimethylamino group and a diethylamino group. Preferred anionically dissociating groups include a quaternary ammonium group and an amino group. Amino groups are classified as a hydrophilic group hereinabove. In the present invention, amino groups can be classified in both the hydrophilic and the anionically dissociating groups.

More preferable polymers of the present invention are, polymers having at least one carboxy group and at least one sulfate group, polymers having at least one quaternary ammonium group and at least one hydroxyethyl group, polymers having at least one quaternary ammonium group and at least one pyrrollidonyl group.

One of the most preferable polymers is described in the Japanese Patent Publication No. Heisei 6-327969 A to Ogawara, et. al, published on Nov. 29, 1994, and filed by Yugen Kaisha Angel Sogo Kenkyusho and the Japanese Patent Publication No. Tokkai 2003-88755 A to Shiraishi, et. al, published on Mar. 25, 2003, and filed by Kabushiki Kaisha Kankyo Joka Kenkyusho.

The polymer of the present invention preferably has a molecular weight of from about 3,000 to about 40,000, more preferably from about 4,000 to about 10,000.

When polymer is used as a malodor removing active, the level of the polymer is from about 0.1% to about 40%, preferably from about 1% to about 20% by weight of the malodor removing material.

Other Malodor Removing Actives

Other malodor removing actives can be organic or inorganic materials, for example, chlorine dioxide, a cyclodextrin, a titanium dioxide, a phtalocyanine, a zinc chloride, a copper compound, an iron compound, a reactive aldehyde, a plant extract, an activated carbon, a zeolite and a mixture thereof

“Cyclodextrin” specifically includes α-, β-, and γ-cyclodextrins, a modified cyclodexrin, a cyclodextrin derivative and a cyclodextrin complex. Preferable cylodextrin is described in U.S. Pat. No. 5,593,670 to Trinh, et al., issued Jan. 14, 1997. Hydroxy-alkyl cyclodextrins and other alkyl-modified cyclodextrins are especially preferred.

A preferred copper compound includes copper pthalocyanine and/or copper chloride. A preferred iron compound includes ferrous sulphate, iron phthalocyanate etc. A preferred reactive aldehyde is 2-Methyl-3-(4-tert-butylphenyl)propane.

A preferred plant extract includes, for example, a catechin and/or a polyphenol.

A preferred activated carbon is provided by, for example, Japan EiviroChemicals LTD., (Osaka, Japan).

A preferred zeolite includes, Zeolite A (Sodium Aluminosilicate), Zeolite MAP, and other commercially-available zeolites which may capture odors.

When the malodor removing actives other than polymers are used as a malodor removing active, the level of them is from about 1% to about 30%, preferably from about 20% to about 30% by weight of the malodor removing material.

(b) Carrier

The malodor removing material of the present invention optionally, but preferably comprises a carrier. The carrier can be any material as long as it can preserve the malodor removing material substantially and sufficiently. The carrier is preferably selected from the group consisting of a gel, beads, a fabric, a nonwoven absorbent material and a mixture thereof. Preferable nonwoven absorbent material is, for example, cellulose, cottons or wood pulp.

More preferably, the carrier of the present invention is a gel. A gel is typically considered to be a colloid in which the disperse phase has combined with the dispersion medium to produce a semisolid material, such as a jelly. The preferable gel can be natural or synthetic gels. Preferred natural gels can be xanthan gum, guar gum, carboxy methyl cellulose or agars. Preferred synthetic gels can be cross-linked polymers such as acrylic based polymers. The gel can be made by combining a dispersion medium such as water, solvent, a solution of active ingredients or mixture of ingredients with the disperse phase such as naturally occurring materials xanthum, agar, alginate, wood pulp, guar or synthetic absorbent polymer such as cross-linked or non cross-linked or partially cross-linked poly acrylic acid, poly acrylamide, poly(ethylene oxide), poly(vinyl alcohol), carboxy methyl cellulose (CMC) and the like. Many more such examples can be found in, for example, Modern Superabsorbent Polymer Technology (Wiley-VCH, 1997), Fredric L. Buchholz and Andrew T. Graham editors.

Gels used for carrier of the present invention can be chemically cross-linked type or physically cross-linked type. Examples of cross-linked type polymers are cross-linked acrylic acid, acrylamide, polyethylene oxide etc. Preferable physically cross-linked type polymers are polyethylene oxides.

It is preferable to use more than one gel. Preferable combination is a combination of gels having a block form and gels having a granule or particle form. Preferable example of the gel having a block form is a polyalkylene oxide such as polyethylene oxide and the gel having a particle form is cross-linked poly acrylic acid.

It is believed that if only from about 4% to about 8% of the block form gel is used, the water absorption capacity may not always be sufficient and the gel may become watery. Watery gels are not very user friendly as water may come out during storage, transportation and/or use. In contrast, a higher percentage of block form gel usage is not economical as well as the gel product may look denser (less transparent and aesthetics is not good). In addition, higher use of the particle form gel may destroy the block form aesthetics of the malodor removing material.

Based on the two forms gel combination, a non watery property and stable block shape is provided.

Considering these, it is preferable that a combination of block form gel and particle form gel is used. The preferred range of block form gel is from about 4% to about 8% by weight of the malodor removing material and the range of the particle form gel is from about 0.2% to about 1.5% by weight of the malodor removing material.

It is to note that in the gel used for carrier in the present invention can be the same as long as such a gel has the functional groups described hereinabove.

(c) Other Ingredients

The malodor removing material of the present invention can further comprise perfume, dye, stabilizer, water, organic solvent (i.e. alcohol, ketone etc.) and/or preservatives.

A highly preferred ingredient in the present invention is a UV protector which is used herein to describe a material which absorbs, blocks and/or reflects UV light so as to reduce UV damage. Specifically, polymer molecules in the gel material may degrade and/or break when exposed to light energy. Many light wavelengths, especially in the UV spectrum are known to affect polymer molecules by breaking and/or weakening the internal chemical bonds between monomers. In the case of gel materials, this may in some cases cause the shape of the gel to become deformed. In the case of gels which are formed into a specific regular shape, such as a block, a circle, a sphere, a star, etc., it may appear that the gel is melting over time. In an extreme case, the shape may be destroyed if excessive breaking of molecules occurring because of exposure to light during manufacture, shipping, storage, and/or use.

The possible detrimental effects of light are even stronger when a transparent or translucent package is used. In a highly preferred embodiment herein current product, a transparent package is used so that the regular shape of the gel material is observable from the outside of the package.

Thus, useful UV protectors include the UV absorber SEESORB™ 101, available from Shipro Kasei Kaisha, Osaka, Japan, which can be absorbed or otherwise incorporated into the gel. SEESORB™ 101 is a benzophenone based UV absorber. Also useful herein are benzo triazole based UV absorbers such as SEESORB 701, also available from Shipro.

Other examples of UV protectors which can be used alone or as a mixture with another UV protectors or with an anti-oxidant include the CYASORB UV series from American Cyanamid Co. (Wayne, N.J., USA) and the Tinogard TL series from Ciba Specialty Cehmicals Co. (Basel, Switzerland). Such UV protectors may be incorporated into any relevant portion of the product, for example, in to the packaging, into or onto the gel, etc.

Anti-oxidants known in the art may also be useful herein to prevent degradation and/or damage to the gel, perfume, and/or other ingredients in the product. While such anti-oxidants are well-known in the art, an example of a preferred anti-oxidant is SEENOX-BCS available from Shipro.

In order to improve UV, perfume, gel, and/or dye stability, it is preferred that the pH of any liquid component be from about 1.5 to about 5, preferably from about 2 to about 4, and more preferably from about 2.5 to about 3.5.

(d) Form

The malodor removing material of the present invention can be formed as a block, liquid, bead chip or sheet. Preferably, the malodor removing material of the present invention has a block form. Preferably the block form is selected from the group consisting of a cube, a sphere, a cone, a triangle, a rectangle, a parallelepiped, a star and a mixture thereof.

When the malodor removing material of the present invention has a block form, the malodor removing material preferably has air between the gel particles, and especially if they are in block form. Air can be incorporated between the gel, and especially block forms by any method, but preferably, vibrations are employed to achieve this outcome. If the air is incorporated between the gel particles, especially for block forms, then light may reflect on the air and may cause a desirable shining effect. Air between the gel particles may also significantly increase the overall MAF. Detailed methods or effects of the air between block forms are described in the Japanese Patent Publication No. Tokkai 2000-212354 A to Misumi et al., published on Aug. 2, 2000 and filed by Kobayashi Seiyaku Kabushiki Kaisha.

(e) Color and Light Absorbance

The malodor removing material of the present invention can be colored in any color which can be adjusted by adding a pigment and/or dye to the malodor removing material. In addition, the malodor removing material may be transparent, translucent or opaque as desired. However, the malodor removing material preferably is either transparent or translucent. If the color of the malodor removing material is transparent or translucent, light may reflect on the malodor removing material and may enhance the desirable shiny effect described above.

(2) Malodor Accessibility Factor (MAF)

The deodorizer of the present invention has MAF of more than about 5×10⁴ ppm²cm²/(g*min), preferably from about 5×10⁴ ppm²cm²/(g*min) to about 15×10⁷ ppm²cm²/(g*min). MAF is, as defined hereinabove, a multiplication of factors consisting of EOSA, AR and AC. Specifically, MAF follows the formula, below:
MAF=(EOSA)*(AR)*(AC)

If the deodorizer has MAF of less than 5×10⁴ ppm²cm²/(g*min), the malodor removing performance may not be sufficient and users may not be able to enjoy the malodor removing benefit. On the other hand, while higher MAF helps in better and faster removal of malodor, the components required to increase the MAF would be highly expensive and would not be commercially feasible to market as a placement type deodorizer product.

(a) EOSA (Effective Open Surface Area of the Malodor Removing Material)

The deodorizer of the present invention has EOSA of from about 60 cm² to about 250 cm², preferably from about 65 cm² to about 200 cm². While high open surface area increases the malodor removal efficacy, the device size may become very large, which could be inconvenient to use as a placement type deodorizer. On the other hand, smaller EOSA may not be able to deliver sufficient malodor removal efficacy.

(b) AR (Absorption Rate of the Malodor Removing Material)

As the air circulation in a room keep changing, the malodor type and intensity coming to the room also change. Thus, in order to maximize users' benefit, the deodorizer needs to absorb the malodor at a faster rate than the air circulation rate. Especially, AR for first 10 minutes from when the deodorizer is placed is an important parameter in deciding how fast the malodor can be removed.

The deodorizer of the present invention has the malodor removing material having AR of more than about 0.35 ppm/min, preferably from about 0.35 ppm/min to about 7 ppm/min, more preferably from about 0.4 ppm/min to about 6 ppm/min.

Measuring AR comprises two steps: preparing ammonia gas for the measurement and measuring AR of the malodor removing material.

Preparing Ammonia Gas

First an air stock with desired ammonia concentration is prepared as follows. A single cock 10 liter Tedlar® Bag (Shibao Shoten, Osaka, Japan) is used for making a stock solution. The Tedlar® Bag is fitted with an open/close valve through which gas can be injected and taken out.

The 10 liter Tedlar® bag is filled with clean air using an air-pump. To make sure that the bag has 10 liters air in it, the air is filled until the bag becomes full and the walls are just tight without any pressure being built in the bag. A gas meter can also be used to confirm the accuracy of the measurement of 10 liters air.

Ammonia gas is obtained from a head space of ammonia solution bottle (500 ml bottle of 30% ammonia solution supplied by Sigma-Aldrich, Japan by using a syringe (Termo Corporation, Tokyo, Japan). Once the headspace of the bottle has equilibriated at room temperature (i.e., after about 24 hours), the syringe is inserted into a mouth of the ammonia bottle and approximately 5-20 ml air above the ammonia solution in the bottle is sucked into the gas syringe.

Ammonia gas is then injected using gas syringe to the Tedlar® Bag in steps of small volumes to achieve a desired initial concentration through the valve. After adding each small volume of ammonia gas, check the concentration with Gastec's standard ammonia (NH₃) detector tube system (Model 1M 003MJ1, supplied by Gastec Corporation, Kanagawa, Japan). The standard system consists of Model GV-100 gas sampling pump and Gastec standard ammonia detector tube.

The measurement is conducted as follows: Break off both end of the detector tube using the built-in tip breaker in the sampling pump. Insert the detector tube in to the sampling pump with the marked side into the sampling pump and align the handle with the mark showing 100 ml, then, insert the other open end of the detector tube into the Tedlar® Bag valve (if the size doesn't fit, then a connecting tube of length 3 cm can be used). Then, opening the Tedlar® Bag valve and pulling the gas sampler handle fully. After waiting for 1 min, read the concentration from the detector tube.

After that, shake the bag well and keep injecting ammonia until the concentration reaches 300 ppm. Then, shake it well and leave it for 5 min and again confirm the concentration using the NH₃ detection tube. For initial concentration of 300 ppm, appropriate NH₃ (ammonia) detector tube needs to be used. For example, for adjusting 300 ppm initial concentration, 1-30 ppm range gas tube which is Gastec No 3 L may be used.

Measuring AR of the Malodor Removing Material

A 1 liter Tedlar® Bag is prepared and one corner of the bag is cut and opened.

0.30 g of malodor removing material sample is prepared and completely spread on a small glass Petri dish. Then, the Petri dish is placed inside the Tedlar® Pak. Any air is pressed out of the bag before sealing.

The cut-opened corner is then sealed completely without any substantial leakage using a heat sealer. Preferable heat sealer is Handy Sealer Manufactured by Iuchi Model 200.

The 1 liter Tedlar® Bag with the sample is filled with NH₃ (concentration 300 ppm) from the 10 liter Tedlar® Bag through a connection tube with a valve. Remove the 10 liter Tedlar® Bag connection and immediately seal the 1 liter Tedlar® Bag by closing the valve.

Leave the bag for 30 seconds and measure the initial concentration of ammonia by NH₃ detector tube gas analyzer (Model 1M 003MJ1, Gastec Corporation, Kanagawa, Japan). A detector tube with 1-30 ppm range is used. Ammonia concentration is measured as a function of time (10 min, 20 min, 30 min and 60 min) using detector tube mentioned above. The absorption rate is taken as the slope of the steepest portion of the curve. Repeat the test three times and take an average of these tests as AR.

A blank (without deodorizer) is also run in the same way. The difference between the blank ammonia concentration data for each data point and that of the corresponding one with the deodorizer is taken as the ammonia concentration data of the deodorizer for each data point.

(c) AC (Absorption Capacity) of the Malodor Removing Material

As explained before, the malodor absorbing molecules needs to be included in to the malodor removing material. While AR helps remove the malodor faster, the malodor removing material capacity is also important in keeping the performance for longer usage times. If the capacity of the malodor removing material gets saturated, then no more malodor can be absorbed into the material and the efficacy will be reduced significantly. Thus, higher capacity is also important in keeping the faster removal rate as more absorbing sites are available for malodor molecules to get absorbed.

The malodor removing material of the present invention has AC of more than about 2500 ppm/g of material, preferably from about 2500 ppm/g to about 90,000 ppm/g, more preferably from about 2800 ppm/g to about 40,000 ppm/g.

AC measurement method is as follows:

Preparing Ammonia Gas

First an air stock with desired ammonia concentration is prepared as follows. A single cock 20 liter Tedlar® Bag is used for making the stock solution. The Tedlar® Bag is fitted with an open/close cock/valve through which gas can be injected and taken out.

20 liter Tedlar® bag is filled with clean air using an air-pump. To make sure that the bag has 20 liters air in it, the air is filled until the bag becomes full and the walls are just tight without any pressure being built in the bag. A gas meter can also be used to confirm the accuracy of the measurement of 20 liters air.

Ammonia gas is obtained from a head space of ammonia solution bottle (500 ml bottle of 30% ammonia solution supplied by Sigma-Aldrich, Japan by using a 60 ml syringe (Termo). The syringe is inserted into a mouth of the ammonia bottle and approximately 20-40 ml air above the ammonia solution in the bottle is sucked into the gas syringe and is then injected to the Tedlar® Bag.

Ammonia gas is then injected by gas syringe to the bag in steps of small volumes to achieve desired initial concentration through the valve. After adding each small volume of ammonia gas, check the concentration with Gastec's NH₃ detector tube (Model 1M 003MJ1. In this case detector tube Gastec No 3 M is used with 50 ml suction. The readings are then multiplied by 2 to get the concentration.

Then, shake the bag well and keep injecting ammonia until the concentration is 1000 ppm. Shake well and leave it for 5 min and again confirm the concentration by the NH₃ detection tube.

Measuring AC of the Malodor Removing Material

A 1 liter Tedlar® Bag is used for AC measurement. One corner of the bag is cut and opened.

0.30 g of malodor removing material sample is prepared and completely spread on a small glass Petri dish. Then, the Petri dish is placed inside the bag through the cut open area.

The cut-opened corner is then sealed completely without any substantial leakage using a heat sealer, Iuchi Model 200. Any air is pressed out of the bag before sealing.

The 1 liter Tedlar® Bag with the sample is filled with NH3 (concentration 1000 ppm) from the 20 L Tedlar® Bag through a connecting tube with a valve. Remove the 20 liter Tedlar® Bag connection and immediately seal the 1 liter Tedlar® Bag by closing the valve. A blank is also run in the same way as above. Measure the concentration of ammonia in the bag after 1 hour by the same method for AR. Blank reading is also measured in the same way. The absolute amount of ammonia absorbed by the malodor removing material is estimated as the difference between removed ammonia concentration in the bag with deodorizer and the removed ammonia concentration in the blank.

The remaining air (with ammonia) is then completely removed from the bag and fresh air with 1000 ppm ammonia is filled in from the same stock gas. The measurements are then taken after 1 hour and the same procedure is followed until there is no difference between the ammonia removed by blank and the deodorizer bag.

Total ammonia absorbed by the deodorizer is taken as the sum of all expressed in ppm/g.

In case if all of the 1000 ppm ammonia is absorbed by the deodorizer in less or near 1 hour time, then either the ammonia concentration should be increased to higher level or the amount of malodor removing material should be decreased so that the remaining ammonia concentration should be +20% of the blank reading. Repeat the test at least two times and take an average of these tests as AC.

To obtain much better malodor removing efficacy, the malodor removing material of the present invention may have a multiplication of AR and AC is more than about 875 ppm²/(g*min), more preferably from about 875 ppm²/(g*min), to about 50,000 ppm²/(g*min), and more preferably from about 1,000 ppm²/(g*min) to about 10,000 ppm²/(g*min).

### EXAMPLE

(1) Preparation for the Malodor Removing Material

A malodor removing active having a carboxylic and an amino group (molecular weight is about 6,000) is prepared. The malodor removing active is mixed with phenoxy ethanol (preservative) in de-ionized water and the pre-mixture is then added to a block type polyethylene oxide (Sumitomo Seika Corporation, Osaka, Japan). Formula is shown in TABLE 1. The mixture is kept for 6 hrs for gel formation. Then, the product is placed on a rectangular tray with 10.75 cm×6.8 cm (73 cm²) open area. The malodor removing material has a cube form and they are prepared to have air between each cube form. The color and light absorbency of the malodor removing material is transparent green.

Also, one marketed product (Marketed product A) is prepared. For measuring AR and AC, 0.30 g of the malodor removing active is taken from the Marketed product and placed on the tray in the same way.

(2) Measurement of EOSA

Based on the definition above, EOSA is measured. The malodor removing material of the present invention has 60 cm² of EOSA, while the Market product A has 56 cm².

(2) Measurement of AR

According to the method described hereinabove, the malodor removing material of the present invention has AR of 0.4 ppm/min. In contract, the market product A has AR of 0.34 ppm/min

(3) Measurement of AC

According to the method described hereinabove, the malodor removing material of the present invention has AC of 3000 ppm/g of AC, while the market product A has AC of 2400 ppm/g.

(4) Calculation of MAF

In the above examples, the deodorizer of the present invention has MAF of 7.2×10⁴ ppm²cm²/(g*min), whereas the Market Product A has MAF of 4.57×10⁴ ppm²cm²/(g*min). The malodor removing material of the present invention has MAF of from about 5×10⁴ ppm²cm²/(g*min) to about 1.5×10⁸ ppm²cm²/(g*min), while the sampled currently marketed product has a MAF of about 2.3×10⁴ ppm²cm²/(g*min) to about 4.6×10⁴ ppm²cm/(g*min).

(5) Odor Removal Efficacy

Odor removal efficacy is also measured using ammonia as the model malodor gas. An odor evaluation room of size 3.3 meters×3.3 meters×2.4 meters is selected at the Toyobo Research Centre, Katata, Japan for the measurement. The temperature of the room is set at 20 degrees C. and at 65% of room humidity. The odor evaluation room is completely secured without any external air or odor entering the room. Thorough cleaning system is used for cleaning the room of any odor after each test. Inlets which can be closed completely after injecting odor are provided at each side.

The Market product A (EOSA of 56 cm²) and the malodor removing material of the present invention with a package (EOSA of 73 cm²) are used. Ammonia gas is injected (ammonia gas collected in syringe as described above) in to the room using a syringe through the inlet. Two small fans are running for 3 minutes to make sure the ammonia is mixed well in the room. The initial concentration of the ammonia in the room is adjusted to 10 ppm by measuring and readjusting as required (ammonia measurement method was described before using Gastec's odor detector tube system).

After adjusting the initial concentration to 10 ppm, the fans are switched-off and the test material is placed in the middle of the room on the floor. Ammonia concentration is measured after 3 hours using the same method as described above. A blank is run in the same way without any deodorizer product in it. The ammonia concentration difference between with Market product A, and the malodor removing material of the present invention and blank is taken as the ammonia removal efficacy data. Then, the difference between Market product A and Blank, and the difference between current invented product and blank have been taken as the ammonia removing efficacy.

The result is shown in TABLE 2. According to TABLE 2, the present invention clearly shows improved malodor removing performance than market products.

TABLE 1

| | Ratio (weight percent of the |
|---|---|
| Ingredients | malodor removing material (%)) |
| Polymer | 10 |
| Water | 80 |
| Gel | 9 |
| Preservative | 1 |
| Total | 100 |

TABLE 2

| | | | | MAF | Odor |
|---|---|---|---|---|---|
| | EOSA | AR | AC | (EOSA) * | Removal |
| Product | (cm²) | (ppm/min) | (ppm/g) | (AR) * (AC) | Efficacy |
| Present | 73 | 0.4 | 3,000 | 87,600 | 300* |
| Invention | | | | | |
| Market | 56 | 0.34 | 2,400 | 45,696 | 100** |
| Product-A | | | | | |

*The odor removal efficacy of the Market Product-A is used as a standard (100).
**For the odor removal efficacy assessment at consumer homes, malodor removal material from the market product was transferred to a tray of EOSA of 73 cm².

All documents cited in the Detailed Description of the Invention are, in relevant part, incorporated herein by reference; the citation of any document is not to be construed as an admission that it is prior art with respect to the present invention. To the extent that any meaning or definition of a term in this written document conflicts with any meaning or definition of the term in a document incorporated by reference, the meaning or definition assigned to the term in this written document shall govern.

While particular embodiments of the present invention have been illustrated and described, it would be obvious to those skilled in the art that various other changes and modifications can be made without departing from the spirit and scope of the invention. It is therefore intended to cover in the appended claims all such changes and modifications that are within the scope of this invention.

What is claimed is:

1. A placement type deodorizer comprising a malodor removing material, wherein the deodorizer has Malodor Accessibility Factor (MAF) of more than about 5×10⁴ ppm²cm²/(g*min) and wherein the malodor removing material comprises a malodor removing active.

2. The deodorizer according to claim 1, wherein the malodor removing active is selected from the group consisting of a polymer, chlorine dioxide, a cyclodextrin, titanium dioxide, a phthalocyanine, a zinc chloride, a copper compound, an iron compound, a reactive aldehyde, a plant extract, an activated carbon, a zeolite and a mixture thereof.

3. The deodorizer according to claim 1, wherein the malodor removing material further comprises a carrier.

4. The deodorizer according to claim 3, wherein the carrier is selected from the group consisting of a gel, a bead, a fabric, a nonwoven absorbent material and a mixture thereof.

5. A placement type deodorizer comprising a malodor removing material, wherein the deodorizer has Malodor Accessibility Factor (MAF) of more than about 5×10⁴ ppm²cm²/(g*min) and the malodor removing material has from about 60 cm² to about 250 cm² of Effective Open Surface Area (EOSA); more than about 0.35 ppm/min of Absorption Rate (AR) and more than about 2500 ppm/g of Absorption Capacity (AC).

6. The deodorizer according to claim 2, wherein the AR×AC is more than about 875 ppm²/(g*min).

7. The deodorizer according to claim 1, wherein the malodor removing material has a block form.

8. The deodorizer according to claim 7, wherein the malodor removing material comprises air between each block form.

9. The deodorizer according to claim 7, wherein the malodor removing material has a block form selected from the group consisting of a cube, a sphere, a cone, a triangle, a rectangle a parallelepiped, a star and a mixture thereof.

10. The deodorizer according to claim 1, wherein the malodor removing material is transparent or translucent.

11. The deodorizer according to claim 1, further comprising a UV protector.

12. The deodorizer according to claim 1, further comprising an anti-oxidant.
